(19) United States
(12) Patent Application Publication
(10) Pub. No.: US 2019/0083573 A1
(43) Pub. Date: Mar. 21, 2019

(54) DESIGNER RECEPTORS FOR MODULATING PAIN AND METHODS OF USE THEREOF

(71) Applicant: The Board of Trustees of the Leland Stanford Junior University, Stanford, CA (US)

(72) Inventors: Shrivats Mohan Iyer, Stanford, CA (US); Scott L. Delp, Stanford, CA (US)

(21) Appl. No.: 16/071,043

(22) PCT Filed: Jan. 25, 2017
(86) PCT No.: PCT/US17/14932

§ 371 Date: Jul. 18, 2018

Related U.S. Application Data
(60) Provisional application No. 62/288,865, filed on Jan. 29, 2016.

(51) Int. Cl. A61K 38/17; A61K 48/00; A61K 31/5513; A61P 29/00

(52) U.S. Cl. CPC A61K 38/1787; A61P 29/00; A61K 31/5513; A61K 48/0083

(57) ABSTRACT

The present disclosure provides a method for modulating pain in a subject comprising activating an exogenous receptor expressed in a target neuron with an effective amount of an agent that specifically activates the exogenous receptor. The present disclosure provides a method for modulating the activity of a neuron comprising activating an exogenous receptor expressed in the neuron by contacting the neuron with an agent that specifically activates the exogenous receptor. The present disclosure provides a method of screening to identify compounds that modulate pain.

### CROSS-REFERENCE

This application claims the benefit of U.S. Provisional Patent Application No. 62/288,865, filed Jan. 29, 2016, which application is incorporated herein by reference in its entirety.

### STATEMENT REGARDING FEDERALLY SPONSORED RESEARCH

This invention was made with Government support under contract NS080954 awarded by the National Institutes of Health. The Government has certain rights in the invention.

### INTRODUCTION

Current pharmacological treatments for chronic pain are marked by lack of efficacy, off-target side-effects, and high addictive risk. Gene therapy approaches to the management of chronic pain are currently in pre-clinical and early-stage clinical trials. Aspects of the present disclosure provide a reversible method to reducing neural activity in a defined nociceptor population that contributes to chronic pain.

### SUMMARY

The present disclosure provides a method of modulating pain in a subject, the method comprising a) delivering a polynucleotide that encodes an activatable exogenous receptor to a target neuron that mediates pain perception in the subject, to express the activatable exogenous receptor in the target neuron; and b) administering to the subject an effective amount of an agent that specifically activates the exogenous receptor to alter the activity of the target neuron, thereby modulating pain in the subject.

In some cases, the present disclosure provides a method of bidirectionally modulating pain in a subject, the method comprising a) delivering a polynucleotide that encodes an activatable exogenous receptor to a target neuron that mediates pain perception in the subject, to express the activatable exogenous receptor in the target neuron; b) administering to the subject an effective amount of an agent that specifically activates the exogenous receptor to alter the activity of the target neuron; c) further delivering a second polynucleotide that encodes a light-activatable polypeptide to the target neuron, to express the light-activatable polypeptide in the target neuron; d) exposing the target neuron to an activating wavelength of light that specifically activates the light activatable polypeptide. In some cases, the activatable exogenous receptor when activated, hyperpolarizes the target neuron, and the light-activatable polypeptide when activated, depolarizes the target neuron, thereby resulting in a method for the bidirectional modulation of pain.

The present disclosure provides a method of modulating activity of a neuron, the method comprising a) contacting the neuron with a polynucleotide that encodes an activatable exogenous receptor, under conditions sufficient to express the activatable exogenous receptor in the neuron; b) contacting the neuron with an agent that specifically activates the activatable exogenous receptor thereby modulating the activity of the neuron.

The present disclosure also provides a screening method to assess whether a test compound modulates pain perception in a subject, the method comprising a) delivering a polynucleotide that encodes an activatable exogenous receptor to a target neuron that mediates pain perception in the subject, to express the exogenous receptor in the target neuron; b) administering to the subject an effective amount of an agent that specifically activates the exogenous receptor; c) administering the test compound; d) determining if the test compound modulates pain perception in the subject.

Kits and systems for practicing the subject methods are also provided.

### BRIEF DESCRIPTION OF THE FIGURES

The skilled artisan will understand that the drawings, described below, are for illustration purposes only. The drawings are not intended to limit the scope of the present teachings in any way.

FIG. 1 depicts the chemogenetic suppression of mechanical withdrawal thresholds.

FIG. 2 depicts the chemogenetic suppression of thermal withdrawal latencies.

FIG. 3 depicts the chemogenetic suppression of optical withdrawal thresholds.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a test compound” refers to one or more test compounds, i.e., a single test compound and multiple test compounds. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a protein, a cell, or an organism, refers to a nucleic acid, protein, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “exogenous” as used herein as applied to a nucleic acid or a protein refers to a nucleic acid or protein that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell. As used herein, the term “endogenous polypeptide” refers to a polypeptide that is normally found in and/or produced by a given bacterium, organism, or cell in nature.

“Recombinant,” as used herein, means that a particular nucleic acid or protein is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

Thus, e.g., the term “recombinant” nucleic acid or “recombinant” protein refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

By “construct” or “vector” is meant a recombinant nucleic acid, generally recombinant DNA, which has been generated for the purpose of the expression and/or propagation of a nucleotide sequence(s) of interest, or is to be used in the construction of other recombinant nucleotide sequences.

The term “transformation” refers to a permanent or transient genetic change induced in a cell following introduction of a nucleic acid (i.e., DNA and/or RNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel et al, Short Protocols in Molecular Biology, 3rd ed., Wiley & Sons, 1995.

The terms “regulatory region” and “regulatory elements”, used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell. As used herein, a “promoter sequence” or “promoter” is a DNA regulatory region capable of binding/recruiting RNA polymerase (e.g., via a transcription initiation complex) and initiating transcription of a downstream (3′ direction) sequence (e.g., a protein coding (“coding”) or non protein-coding (“non-coding”) sequence. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein), it may be a spatially restricted promoter (e.g., tissue specific promoter, cell type specific promoter, etc.), and/or it may be a temporally restricted promoter (e.g., the promoter is in the “ON” state or “OFF” state during specific stages of embryonic development or during specific stages of a biological process, e.g., hair follicle cycle in mice).

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non protein coding sequence, e.g., a sequence encoding a Shh protein; and the like) if the promoter affects its transcription and/or expression.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

In further describing the subject invention, methods for modulating pain are described first in greater detail. Next, screening methods of interest are described. Compositions and kits are also described.

### Methods for Modulating Pain

As summarized above, aspects of the invention include methods for modulating pain in a subject, wherein a polynucleotide that encodes an activatable exogenous receptor is delivered to a target neuron that mediates pain perception in the subject, to express the activatable exogenous receptor in the target neuron.

As used here, the term “modulating pain” refers to the modulation (e.g., inhibition or diminishment) of pain or the perception of pain in a given subject and includes absence from pain sensations as well as states of reduced or absent sensitivity to pain stimuli. Such states of reduced or absent pain perception are induced by “Pain perception” refers to pain that a given subject feels in response to a stimuli.

In general terms, the subject methods involve chemogenetic techniques that can provide for efficient in vivo delivery of a particular polynucleotide to a target neuron. The target neurons then can express the polynucleotide as an activatable exogenous receptor which is capable of modulating the activity of the neuron (e.g., as described herein) when activated. The activatable exogenous receptor can be a synthetic G protein-coupled receptor (GPCR) (e.g., a DREADD) that may be activated through systemic or local administration of ligands which specific bind to the receptor (e.g., clozapine-N-oxide). In some cases, the ligand specifically activates the exogeneous GPCR that is delivered and expressed in the target neurons over other endogenous GPCR receptors.

The effect of the subject methods depends on the exogenous GPCR that is expressed. In some cases, upon activation, the exogenous GPCR may alter the activity of a target neuron, e.g., change the membrane polarization properties of the target neuron, and/or change the level of presynaptic neurotransmitter (e.g., glutamate) release.

Methods for modulating pain in a subject can also include administering to the subject an effective amount of an agent that specifically activates the exogenous receptor, resulting in the modulation of activity of the target neuron.

In some cases, methods for modulating pain in a subject comprise delivering a polynucleotide that encodes an activatable exogenous receptor to a target neuron that mediates pain perception in the subject, to express the activatable exogenous receptor in the target neuron, and administering to the subject an effective amount of an agent (e.g., a synthetic small molecule) that specifically activates the exogenous receptor to modulate the activity of the target neuron.

As used here, the term “modulating the activity” of a given target cell (e.g., neuron) refers to changing the activity level of a cell function. For example, altering the activity of a target neuron may include changing the membrane potential of a neuron, wherein the membrane potential of a neuron is important for its function (e.g., action potential firing). In some cases, the activity of the neuron is altered such that the membrane potential is increased (e.g., hyperpolarized). In some cases, the activity of the neuron is altered such that the membrane potential is decreased below a threshold potential, resulting in an action potential (e.g., depolarized).

In some cases, the polynucleotide encodes an activatable exogenous receptor that when activated results in the hyperpolarization of the target neuron (i.e., neuronal silencing). In other cases the polynucleotide encodes an activatable exogenous receptor that when activated results in the depolarization of the target neuron (i.e., neuronal firing). In other cases, the polynucleotide encodes an activatable exogenous receptor that when activated results in reduced presynaptic glutamate release. In methods where activation of the activatable exogenous receptor results in hyperpolarization of the target neuron or reduced presynaptic glutamate release, wherein the target neuron is a neuron that mediates pain perception (e.g., an unmyelinated primary afferent neuron), the method decreases the level of pain perceived by the subject.

In some cases, methods for modulating pain in a subject further comprise delivering a second polynucleotide that encodes a light-activatable polypeptide to the target neuron. In some cases, activation of the light-activatable polypeptide in the target neuron depolarizes the target neuron, and activation of the activatable exogenous receptor in the target neuron inactivates the target neuron, resulting in the bidirectional control of pain in a subject.

### Polynucleotides for Delivery to a Target Neuron

Aspects of the present disclosure include polynucleotides which can be delivered to a target neuron. The subject polynucleotides can encode target polypeptide sequences for expression in the target neuron. Any convenient polynucleotides which encode a target polypeptide sequence can be adapted for use in the methods described hereon. In some instances, the polynucleotide comprises a nucleic acid sequence that encodes an activatable exogenous receptor. In certain instances, the polynucleotide comprises a nucleic acid sequence that encodes a light-activatable polypeptide.

### Activatable Exogenous Receptors

A polynucleotide of the present disclosure can comprise a nucleic acid sequence that encodes an activatable exogenous receptor. As used here, the term “exogenous receptor” refers to a receptor that is not normally or naturally found in and/or produced by a given cell. As used here, the term “activatable”, for example as used in an “activatable exogenous receptor”, refers to a non-naturally found receptor in the given cell that is activated upon binding to a ligand or small molecule. In some cases, activation of an exogenous receptor results in activation of downstream signaling.

Any convenient activatable exogenous receptors can be adapted for use in the subject methods. In some cases, the activatable exogenous receptor is a Designer Receptor Exclusively Activated by Designer Drug (DREADD). An activatable exogenous receptor (e.g., DREADD) can be generated from a G-protein coupled receptor having separable domains for: 1) natural ligand (e.g., peptide ligand or biogenic amine) binding; 2) synthetic small molecule binding; and 3) G-protein interaction. In general, any convenient G-protein coupled receptor may be engineered into an activatable exogenous receptor of the present disclosure. Activatable exogenous receptors of interest, include but are not limited to, a DREADD such as a Receptor Activated Superiorly by Synthetic Ligand (RASSL). RASSL receptors of interest which may be adapted for use in the subject methods, include but are not limited to, those described extensively in U.S. Pat. No. 6,518,480, the disclosure of which is herein incorporated by reference.

A DREADD can be of one of several classes of modified G-protein coupled receptors. The specificity of G-protein activation is mediated at the level of the G-protein coupled receptor. Each G-protein coupled receptor activates a distinct G protein subset (class) which elicits different physiologic effects depending on the cell type. Of the four major classes of G proteins (Gₛ, Gᵢ, G_{q} and G₁₂), each class is associated with cell proliferation in a different cell type. As such, a DREADD can be generated from a variety of G-protein coupled receptors that are coupled to any of the G_{q}, Gᵢ, Gₛ, G₁₂ proteins and mediate various kinds of signaling.

The expression of an activatable exogenous receptor (e.g., a DREADD) in a target cell may allow for transformed cells to be selectively activated. In some cases, the activatable exogenous receptor is a modified native endogenous receptor that is naturally expressed by the target cell. The activatable exogenous receptor (e.g., DREADD) can be a modified G-protein coupled receptor having decreased binding affinity for a selected natural (i.e., endogenous) ligand (relative to binding of the selected ligand by a wild-type G-protein coupled receptor), but having normal, near normal, or enhanced binding affinity for a synthetic small molecule. Thus, activatable exogenous receptor-mediated activation of transformed cells does not occur to a significant extent in the present of the selected natural ligand, but responds significantly upon exposure to a synthetic small molecule.

In some cases, an activatable exogenous receptor is a DREADD that is a modified G-protein coupled receptor. In some instances, the DREADD comprises changes and/or modifications in the amino acid sequence of a native G-protein coupled receptor to provide for a desired DREADD characteristics (e.g., decreased natural ligand binding relative to synthetic small molecule binding) without significantly adversely affecting the ability of the DREADD to bind a synthetic small molecule ligand and elicit a desired cellular response.

As used herein, a “small molecule” refers to a low molecular weight organic compound that may help regulate a biological process. A small molecule may have a molecular weight of less than about 900 daltons and may have a size of about 10 nm. A “synthetic small molecule” refers to any small molecule that is made by chemical synthesis. A synthetic small molecule may be made to imitate a naturally occurring small molecule.

As used herein, “natural ligand” refers to a naturally-occurring substance that forms a complex with a biomolecule (e.g., protein) to serve a biological purpose. For example, a natural ligand of a specific receptor refers to a ligand that binds to the receptor to achieve a biological outcome.

Any convenient synthetic small molecules and ligands (e.g., agonists or antagonists) that can selectively activate an activatable exogenous receptor may be utilized in the subject methods. Small molecules of interest include, but are not limited to, those described by Freidinger (Prog. Drug. Res. 1993. 40:33-98), the disclosure of which is herein incorporated by reference. In some cases, the synthetic small molecule is a high affinity agonist or antagonist for the activatable exogenous receptor (e.g., DREADD). Binding of the synthetic small molecule by the activatable exogenous receptor is relatively enhanced compared to binding of the synthetic small molecule by the native endogenous receptor. In certain instances, the activatable exogenous receptor is derived from a target native endogenous receptor that is capable of modulating the activity of the neuron, e.g., by mutation of one or more amino acid residues of the receptor to provide a mutant receptor that is activatable.

In some cases, binding of the synthetic small molecule (e.g., clozapine-N-oxide) by the activatable exogenous receptor (e.g., DREADD) is enhanced by at least 2-fold, 3-fold, 4-fold, 5-fold, 10-fold, 20-fold, 25-fold, 50-fold, 75-fold, 100-fold, 1000-fold or even more relative to binding of the synthetic small molecule by a native endogenous receptor (e.g., an endogenous parent receptor from which the exogenous receptor was derived). In some cases, the synthetic small molecule has an affinity for the activatable exogenous receptor of about 0.3 μM or less, e.g., about 100 nM or less, about 30 nM or less, about 10 nM or less, about 3 nM or less, about 1 nM or less, or about 500 pM or less. In some instances, the synthetic small molecule has a selectivity for the activatable exogenous receptor over any endogenous receptor, such as a selectivity of 2× or more, such as 5× or more, 10× or more, 20× or more, 50× or more, 100× or more, 300× or more, 1000× or more or even more, as measured via any convenient biological property of the receptor, such as binding affinity, receptor activation, and the like.

In some cases, a synthetic small molecule specifically binds to the activatable exogenous receptor and has minimal, negligible, no significant off-target effects. As such, administering a synthetic small molecule will have the effect of selective binding to the activatable exogenous receptor expressed only in transformed target cells. In some cases the synthetic small molecule is clozapine-N-oxide (CNO). In some cases, the synthetic small molecule is a derivative of CNO, e.g., N-desmethylclozapine. Small molecules of interest which find use in the subject methods include, but are not limited to, those compounds described by U.S. Pat. No. 8,771,972 (which describes various clozapine derivatives), perlapine, and compounds described by Chen et al, ACS Chem. Neuro., 2015. 6(3):476-484.

In some instances, an activatable exogenous receptor is a DREADD that is activated by a synthetic small molecule. In some cases, the synthetic small molecule (e.g., CNO) selectively activates the DREADD, that upon activation, results in a cellular G-protein coupled physiological response associated with a disease or condition. For example, a method of modulating pain in the present disclosure can be mediated by exposing DREADD-expressing primary afferent neurons of a subject, to a synthetic small molecule which may result in the activation of a G-protein signaling cascade that leads to decreased cyclic AMP (cAMP) levels and resultant neuronal inhibition.

In some cases, activation of an activatable exogenous receptor modulates cAMP levels in a target cell. In some cases, activation of the activatable exogenous receptor decreases cAMP levels in a target cell (e.g., neuron) to inhibit target cell activity. In some cases, activation of the activatable exogenous receptor decreases cAMP levels in a target neuron by at least about 10%, e.g., by at least about 12%, by at least about 14%, by at least about 16%, by at least about 18%, by at least about 20%, by at least about 25%, by at least about 30%, by at least about 35%, by at least about 40%, by at least about 45%, by at least about 50%, by at least about 55%, by at least about 60%, by at least about 65%, by at least about 70%, by at least about 80%, by at least about 90%, or more.

A target neuron expressing an activatable exogenous receptor, upon exposure to a synthetic small molecule that specifically binds the exogenous receptor, may become hyperpolarized. A target neuron expressing an activatable exogenous receptor, upon exposure to a synthetic small molecule that specifically binds the exogenous receptor, may become depolarized. In some cases, a target neuron expressing an activatable exogenous receptor, upon exposure to a synthetic small molecule that specifically binds the exogenous receptor, results in reduced presynaptic glutamate release. In certain cases, a target neuron expressing an activatable exogenous receptor, upon exposure to a synthetic small molecule that specifically binds the exogenous receptor, results in enhanced presynaptic glutamate release.

A subject polynucleotide may comprise a nucleic acid sequence that encodes for a DREADD. In some cases, the DREADD is an engineered muscarinic receptor that is selectively activated by the synthetic small molecule clozapine-N-oxide. Exposure of DREADD-expressing target cells to CNO can result in activation or inhibition of the target cells.

In some cases, a DREADD suitable for use is hM3D(G_{q}) which is a G_{q}-coupled modified human muscarinic receptor 3. Human muscarinic receptor 3 has the amino acid sequence set forth below:

(SEQ ID NO: 1)
MTLHNNSTTSPLFPNISSSWIHSPSDAGLPPGTVTHFGSYNVSRAAGNF
SSPDGTTDDPLGGHTVWQVVFIAFLTGILALVTIIGNILVIVSFKVNKQ
LKTVNNYFLLSLACADLIIGVISMNLFTTYIIMNRWALGNLACDLWLAI
DYVASNASVMNLLVISFDRYFSITRPLTYRAKRTTKRAGVMIGLAWVIS
FVLWAPAILFWQYFVGKRTVPPGECFIQFLSEPTITFGTAIAAFYMPVT
IMTILYWRIYKETEKRTKELAGLQASGTEAETENFVHPTGSSRSCSSYE
LQQQSMKRSNRRKYGRCHFWFTTKSWKPSSEQMDQDHSSSDSWNNNDAA
ASLENSASSDEEDIGSETRAIYSIVLKLPGHSTILNSTKLPSSDNLQVP
EEELGMVDLERKADKLQAQKSVDDGGSFPKSFSKLPIQLESAVDTAKTS
DVNSSVGKSTATLPLSFKEATLAKRFALKTRSQITKRKRMSLVKEKKAA
QTLSAILLAFIITWTPYNIMVLVNTFCDSCIPKTFWNLGYWLCYINSTV
NPVCYALCNKTFRTTFKMLLLCQCDKKKRRKQQYQQRQSVIFHKRAPEQ
AL.

As used herein, a hM3D(G_{q}) DREADD encompasses an amino acid sequence having at least 75%, at least 80%, at least 85%, at least 90%, at least 95%, at least 98%, at least 99%, or 100%, amino acid sequence identity to the amino acid sequence of the human muscarinic receptor 3 set forth in SEQ ID NO:1.
In some cases, the DREADD hM3D(G_{q}) has the amino acid sequence set forth below:

(SEQ ID NO: 2)
MTLHNNSTTSPLFPNISSSWIHSPSDAGLPPGTVTHFGSYNVSRAAGNF
SSPDGTTDDPLGGHTVWQVVFIAFLTGILALVTIIGNILVIVSFKVNKQ
LKTVNNYFLLSLACADLIIGVISMNLFTTYIIMNRWALGNLACDLWLAI
DCVASNASVMNLLVISFDRYFSITRPLTYRAKRTTKRAGVMIGLAWVIS
FVLWAPAILFWQYFVGKRTVPPGECFIQFLSEPTITFGTAIAGFYMPVT
IMTILYWRIYKETEKRTKELAGLQASGTEAETENFVHPTGSSRSCSSYE
LQQQSMKRSNRRKYGRCHFWFTTKSWKPSSEQMDQDHSSSDSWNNNDAA
ASLENSASSDEEDIGSETRAIYSIVLKLPGHSTILNSTKLPSSDNLQVP
EEELGMVDLERKADKLQAQKSVDDGGSFPKSFSKLPIQLESAVDTAKTS
DVNSSVGKSTATLPLSFKEATLAKRFALKTRSQITKRKRMSLVKEKKAA
QTLSAILLAFIITWTPYNIMVLVNTFCDSCIPKTFWNLGYWLCYINSTV
NPVCYALCNKTFRTTFKMLLLCQCDKKKRRKQQYQQRQSVIFHKRAPEQ
AL

As used herein, a hM3D(G_{q}) DREADD encompasses an amino acid sequence having at least 75%, at least 80%, at least 85%, at least 90%, at least 95%, at least 98%, at least 99%, or 100%, amino acid sequence identity to the amino acid sequence set forth in SEQ ID NO:2.
Exposure of hM3D(G_{q})-expressing target cells to CNO results in the activation of G_{q}-mediated signaling. In some cases, hM3D(G_{q}) is expressed in a neuron, and upon exposure to CNO, results in neuronal burst firing. In some cases, hM3D(G_{q}) is expressed in a neuron, and upon exposure to CNO, results in depolarization of the neuronal cell membrane.

In some cases, a DREADD suitable for use is hM4D(Gᵢ) which is a Gᵢ-coupled modified human muscarinic receptor 4. Human muscarinic receptor 4 has the amino acid sequence set forth below:

(SEQ ID NO: 3)
MANFTPVNGSSGNQSVRLVTSSSHNRYETVEMVFIATVTGSLSLVTVVG
NILVMLSIKVNRQLQTVNNYFLFSLACADLIIGAFSMNLYTVYIIKGYW
PLGAVVCDLWLALDYVVSNASVMNLLIISFDRYFCVTKPLTYPARRTTK
MAGLMIAAAWVLSFVLWAPAILFWQFVVGKRTVPDNQCFIQFLSNPAVT
FGTAIAAFYLPVVIMTVLYIHISLASRSRVHKHRPEGPKEKKAKTLAFL
KSPLMKQSVKKPPPGEAAREELRNGKLEEAPPPALPPPPRPVADKDTSN
ESSSGSATQNTKERPATELSTTEATTPAMPAPPLQPRALNPASRWSKIQ
IVTKQTGNECVTAIEIVPATPAGMRPAANVARKFASIARNQVRKKRQMA
ARERKVTRTIFAILLAFILTWTPYNVMVLVNTFCQSCIPDTVWSIGYWL
CYVNSTINPACYALCNATFKKTFRHLLLCQYRNIGTAR.

As used herein, a hM4D(Gᵢ) DREADD encompasses an amino acid sequence having at least 75%, at least 80%, at least 85%, at least 90%, at least 95%, at least 98%, at least 99%, or 100%, amino acid sequence identity to the amino acid sequence of the human muscarinic receptor 3 set forth in SEQ ID NO:3.
In some cases, the DREADD hM4D(Gᵢ) has the amino acid sequence set forth below:

(SEQ ID NO: 4)
MANFTPVNGSSGNQSVRLVTSSSHNRYETVEMVFIATVTGSLSLVTVVG
NILVMLSIKVNRQLQTVNNYFLFSLACADLIIGAFSMNLYTVYIIKGYW
PLGAVVCDLWLALDCVVSNASVMNLLIISFDRYFCVTKPLTYPARRTTK
MAGLMIAAAWVLSFVLWAPAILFWQFVVGKRTVPDNQCFIQFLSNPAVT
FGTAIAGFYLPVVIMTVLYIHISLASRSRVHKHRPEGPKEKKAKTLAFL
KSPLMKQSVKKPPPGEAAREELRNGKLEEAPPPALPPPPRPVADKDTSN
ESSSGSATQNTKERPATELSTTEATTPAMPAPPLQPRALNPASRWSKIQ
IVTKQTGNECVTAIEIVPATPAGMRPAANVARKFASIARNQVRKKRQMA
ARERKVTRTIFAILLAFILTWTPYNVMVLVNTFCQSCIPDTVWSIGYWL
CYVNSTINPACYALCNATFKKTFRHLLLCQYRNIGTAR.

In some cases, a hM4D(Gᵢ) DREADD encompasses an amino acid sequence having at least 75%, at least 80%, at least 85%, at least 90%, at least 95%, at least 98%, at least 99%, or 100%, amino acid sequence identity to the amino acid sequence of the human muscarinic receptor 3 set forth in SEQ ID NO:4.
Exposure of hM4D(Gᵢ)-expressing target cells to CNO results in the activation of Gᵢ-mediated signaling. In some cases, hM4D(Gᵢ) is expressed in a neuron, and upon exposure to CNO, results in neuronal silencing. In some cases, hM4D(Gᵢ) is expressed in a neuron, and upon exposure to CNO, results in hyperpolarization of the neuronal cell membrane.

In other cases, a DREADD suitable for use is Gₛ-DREADD which is a Gₛ-coupled modified rat muscarinic receptor 3. Rat muscarinic receptor 3 has the amino acid sequence set forth below:

(SEQ ID NO: 5)
MTLHSNSTTSPLFPNISSSWVHSPSEAGLPLGTVTQLGSYNISQETGNF
SSNDTSSDPLGGHTIWQVVFIAFLTGFLALVTIIGNILVIVAFKVNKQL
KTVNNYFLLSLACADLIIGVISMNLFTTYIIMNRWALGNLACDLWLSID
YVASNASVMNLLVISFDRYFSITRPLTYRAKRTTKRAGVMIGLAWVISF
VLWAPAILFWQYFVGKRTVPPGECFIQFLSEPTITFGTAIAAFYMPVTI
MTILYWRIYKETEKRTKELAGLQASGTEAEAENFVHPTGSSRSCSSYEL
QQQGVKRSSRRKYGRCHFWFTTKSWKPSAEQMDQDHSSSDSWNNNDAAA
SLENSASSDEEDIGSETRAIYSIVLKLPGHSSILNSTKLPSSDNLQVSN
EDLGTVDVERNAHKLQAQKSMGDGDNCQKDFTKLPIQLESAVDTGKTSD
TNSSADKTTATLPLSFKEATLAKRFALKTRSQITKRKRMSLIKEKKAAQ
TLSAILLAFIITWTPYNIMVLVNTFCDSCIPKTYWNLGYWLCYINSTVN
PVCYALCNKTFRTTFKTLLLCQCDKRKRRKQQYQQRQSVIFHKRVPEQA
L.

As used herein, the Gₛ-DREADD encompasses an amino acid sequence having at least 75%, at least 80%, at least 85%, at least 90%, at least 95%, at least 98%, at least 99%, or 100%, amino acid sequence identity to the amino acid sequence of the rat muscarinic receptor 3 set forth in SEQ ID NO:5. Exposure of Gₛ-DREADD expressing target cells to CNO results in the activation of Gₛ-mediated signaling. In some cases, Gₛ-DREADD is expressed in a neuron and upon exposure to CNO, leads to an increase in cAMP.

### Light-Activatable Polypeptides

In some instances, a subject polynucleotide comprises a nucleic acid sequence that encodes a light-activatable polypeptide. In some cases, a light-activatable polypeptide is a light-activatable cation channel. In some cases, a target cell that expresses a light-activatable polypeptide can be activated or inhibited upon exposure to light of varying wavelengths. In some cases, a target cell that expresses a light-activatable polypeptide is a neuronal cell that expresses a light-activatable polypeptide, and upon exposure to light of varying wavelengths, results in depolarization or polarization of the neuron.

In some cases, a light-activatable polypeptide mediates a hyperpolarizing current in the target cell it is expressed in when the cell is illuminated with light. Non-limiting examples of light-activatable polypeptides capable of mediating a hyperpolarizing current can be found, e.g., in International Patent Application No. PCT/US2011/028893; U.S. Pat. No. 9,175,095. Non-limiting examples of hyperpolarizing light-activatable polypeptides include NpHr, eNpHr2.0, eNpHr3.0, eNpHr3.1 or GtR3. In some cases, a light-activatable polypeptide mediates a depolarizing current in the target cell it is expressed in when the cell is illuminated with light. Non-limiting examples of depolarizing light-activatable polypeptides include “C1V1”, ChR1, VChR1, ChR2. Additional information regarding other light-activated cation channels, anion pumps, and proton pumps can be found in U.S. Patent Application Publication Nos: 2009/0093403; and International Patent Application No: PCT/US2011/028893.

In one embodiment, the light-activatable polypeptide can be activated by light having a wavelength of about 560 nm. In one embodiment, the light-activatable polypeptide can be activated by red light. In another embodiment, the light-activatable polypeptide can be activated by light having a wavelength of about 630 nm. In other embodiments, the light-activatable polypeptide can be activated by violet light. In one embodiment, light-activatable polypeptide can be activated by light having a wavelength of about 405 nm. In other embodiments, the light-activatable polypeptide can be activated by green light. A person of skill in the art would recognize that each light-activatable polypeptide will have its own range of activating wavelengths.

In some embodiments, the light-activatable polypeptides are activated by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 sec, about 1.25 sec, about 1.5 sec, or about 2 sec, inclusive, including any times in between these numbers. In some embodiments, the light-activatable polypeptides are activated by light pulses that can have a light power density of any of about 0.05 mW/mm², about 0.1 mW/mm², about 0.25 mW/mm², about 0.5 mW/mm², about 0.75 mW/mm², about 1 mW/mm², about 2 mW/mm², about 3 mW/mm², about 4 mW/mm², about 5 mW/mm², about 6 mW/mm², about 7 mW/mm², about 8 mW/mm², about 9 mW/mm², about 10 mW/mm², about 11 mW/mm², about 12 mW/mm², about 13 mW/mm², about 14 mW/mm², about mW/mm², about 16 mW/mm², about 17 mW/mm², about 18 mW/mm², about 19 mW/mm², about 20 mW/mm², about 21 mW/mm², about 22 mW/mm², about 23 mW/mm², about 24 mW/mm², or about 25 mW/mm², inclusive, including any values between these numbers. In some embodiments the neuronal cell can be a primary afferent neuron that mediates the perception of pain in a subject. In some cases, the neuronal cell is an unmyelinated primary afferent neuron. In other cases the neuronal cell is a nociceptor. In some cases the neuronal cell is any neuronal cell that mediates the perception of pain in a subject.

### Promoters and Vectors

Aspects of the present invention disclose expression cassettes and/or vectors comprising the above-described polynucleotides. Suitably, the polynucleotides can comprise promoters operably linked to the region of the polynucleotide that encodes e.g., the activatable exogenous receptor or the light-activatable polypeptide. Virtually any promoter capable of driving these polynucleotides can be used.

Targeted expression can be accomplished using a cell specific promoter. Examples of cell specific promoters are promoters for somatostatin, parvalbumin, GABAα6, L7, and calbindin. Other cell specific promoters can be promoters for kinases such as PKC, PKA, and CaMKII; promoters for other ligand receptors such as NMDAR1, NMDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Specifically, where expression of a subject polynucleotide in a primary afferent neuron is desired, a human calmodulin-dependent kinase II alpha (CaMKIIα) promoter may be used. In some cases, the human synapsin 1 promoter or the human Thy1 promoter may be used. A person of skill in the art will recognize that various promoters drive expression in various cell types, and will be able to decide on which promoter to use for their desired outcome.

Also provided herein are vectors comprising the polynucleotides disclosed herein encoding a C1V1 chimeric polypeptide or any variant thereof. The vectors that can be administered according to the present invention also include vectors comprising a polynucleotide which encodes an RNA (e.g., RNAi, ribozymes, miRNA, siRNA) that when transcribed from the polynucleotides of the vector will result in the accumulation of light-activated chimeric proteins on the plasma membranes of target animal cells. Vectors which may be used include, without limitation, lentiviral, HSV, and adenoviral vectors. Lentiviruses include, but are not limited to HIV-1, HIV-2, SIV, FIV and EIAV. Lentiviruses may be pseudotyped with the envelope proteins of other viruses, including, but not limited to VSV, rabies, Mo-MLV, baculovirus and Ebola. Such vectors may be prepared using standard methods in the art.

In some embodiments, the vector is a recombinant adeno-associated virus (AAV) vector. AAV vectors are DNA viruses of relatively small size that can integrate, in a stable and sitespecific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV could infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Virol., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, UK (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

For the animal cells described herein, it is understood that one or more vectors may be administered to neural cells, heart cells, or stem cells. If more than one vector is used, it is understood that they may be administered at the same or at different times to the animal cell.

### Methods of Delivery

Delivery of a polynucleotide or agent of the present disclosure to a target cell in a subject can be accomplished using a number of different delivery devices, methods and systems. One such delivery device for delivering an agent (e.g., synthetic small molecule) to a target cell is an implantable drug-infusion pump. In some cases, delivery methods (e.g., injection) of a polynucleotide or agent include intraneural delivery (e.g., intraneural injection), intrathecal, topical, intradermal, intra-dorsal root ganglion, or intravenous delivery. In cases where the polynucleotide is packaged into an AAV vector, delivery methods may include intraneural viral delivery, intrathecal viral delivery, topical viral delivery, intradermal viral delivery, intra-dorsal root ganglion viral delivery, intravenous viral delivery.

### Target Cells and Subjects

One aspect of the present disclosure is a cell that expresses an activatable exogenous receptor, and specifically a cell that expresses a Designer Receptor Exclusively Activated by Designer Drug (DREADD). Another aspect is an activatable exogenous receptor-expressing cell that also expresses a light-activatable polypeptide.

A cell that expresses an activatable exogenous receptor can be created using a vector including a DNA expression vector, a virus or an organism. In some aspects, vectors include lentiviruses and retroviruses. In some cases, a cell that expresses an activatable exogenous receptor is created by previously discussed methods of polynucleotide delivery.

Cells of any origin are candidate cells for transfection or infection with a DREADD of the present disclosure. Non-limiting examples of candidate cell types include connective tissue elements such as fibroblast, skeletal tissue (bone and cartilage), skeletal, cardiac and smooth muscle, epithelial tissues (e.g. liver, lung, breast, skin, bladder and kidney), neural cells (glia and neurons), endocrine cells (adrenal, pituitary, pancreatic islet cells), bone marrow cells, melanocytes, and many different types of hematopoetic cells. Suitable cells can also be cells representative of a specific body tissue from a subject. The types of body tissues include, but are not limited, to blood, muscle, nerve, brain, heart, lung, liver, pancreas, spleen, thymus, esophagus, stomach, intestine, kidney, testis, ovary, hair, skin, bone, breast, uterus, bladder, spinal cord and various kinds of body fluids.

In some aspects, a cell that expresses an activatable exogenous receptor is a cell that mediates the perception of pain in a subject. Such a cell can be a nociceptor (e.g., a primary afferent nociceptor), a sensory neuron that responds to potentially damaging stimuli by sending signals to the spinal cord and brain. Of particular interest are mammalian nociceptors that are found in areas of the body that can sense noxious stimuli either externally or internally. Non-limiting examples of locations where nociceptors can be found include skin (e.g., cutaneous nociceptors), cornea, mucosa, muscle, joint, bladder, gut. Neuronal cell bodies of nociceptors are located in the dorsal root ganglia or trigeminal ganglia. In some cases, the nociceptor is a primary afferent neuron. In some cases the nociceptor is an unmyelinated primary afferent neuron that mediates the perception of pain in a subject. Suitable cells for use in methods of the present disclosure include any cell that mediates the perception of pain in a subject.

Cells suitable for use in a subject method include cells of a variety of subject hosts. Generally such subject hosts are “mammals” or “mammalian”, where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In many aspects, the subject host will be a human.

Subjects of the present disclosure can include a variety of subjects capable of perceiving pain, with assays known in the art able to measure the perception of pain by the subject. Generally, such subjects are “mammals” or “mammalian”, where these terms are used broadly to describe organisms which are within the class mammalia, including the orders carnivore (e.g., dogs and cats), rodentia (e.g., mice, guinea pigs and rats), and primates (e.g., chimpanzees and monkeys). In some cases, non-mammalian subjects can be used (e.g., Drosophila, C. elegans and zebrafish).

### Methods of Assessing Pain

Aspects of the disclosure include methods for modulating pain in a subject, wherein a polynucleotide that encodes an activatable exogenous receptor is delivered to a target neuron that mediates pain perception in the subject, to express the activatable exogenous receptor in the target neuron. Methods for modulating pain in a subject may also include administering to the subject an effective amount of an agent that specifically activates the exogenous receptor, resulting in the altering of activity of the target neuron.

Subject methods include methods for assessing whether a subject perceives pain. In some cases, delivering a polynucleotide that encodes an activatable exogenous receptor to a target neuron that mediates pain perception in a subject, and administering to the subject an effective amount of an agent that specifically activates the exogenous receptor results in enhanced or reduced levels of pain perception. The perception of pain in a subject may be assayed by various methods known in the art. For example, a formalin assay may be performed. Other assays, e.g., the Von Frey assay, the Hargreaves assay and thermal assays may be performed to assess the perception of pain in a subject. Thermal assays can include, e.g., a tail withdrawal test, a hot-plate test, a tail flick test.

### Methods of Screening

The present disclosure provides screening methods to assess whether a test compound modulates pain perception in a subject. In some cases, a screening method of the present disclosure comprises a) delivering a polynucleotide that encodes an activatable exogenous receptor to a target neuron that mediates pain perception in the subject, to express the receptor in the target neuron; b) administering to the subject an effective amount of an agent that specifically activates the exogenous receptor; c) administering the test compound; and d) determining if the test compound modulates pain perception in the subject.

In some cases, a screening method comprises delivering a polynucleotide that encodes an activatable exogenous receptor to a target neuron that mediates pain perception in the subject, wherein the activatable exogenous receptor when activated by an agent (e.g., synthetic small molecule), hyperpolarizes the target neuron and results in enhanced perception of pain in the subject, and wherein administering a test compound to the subject reduces perception of pain in the subject. Such a test compound represents a class of test compounds that reduces pain. In some cases, the activatable exogenous receptor is a DREADD that specifically binds a synthetic small molecule agent. In some cases, the activatable exogenous receptor is a modified human muscarinic receptor 3, and the agent is clozapine-N-oxide (CNO).

In some cases, a screening method comprises a) delivering a polynucleotide that encodes a modified human muscarinic receptor 3 to a target neuron that mediates pain perception in the subject, to express the receptor in the target neuron, wherein the target neuron is a primary afferent nociceptor; b) administering to the subject an effective amount of clozapine-N-oxide that specifically activates the modified human muscarinic receptor 3; c) administering the test compound; and d) determining if the test compound modulates pain perception in the subject.

In some cases, a screening method employs the use of a transgenic subject, wherein the subject stably expresses an activatable exogenous receptor (e.g., human muscarinic receptor 3). In some cases, the agent that specifically binds the exogenous receptor (e.g., CNO) and the test compound can be administered at the same time. In some cases, a mixture comprising the agent and test compound is administered. In some cases the agent and test compound can be administered sequentially, i.e., the test compound is administered a duration after the agent is administered (e.g., a duration long enough such that the agent has activated the exogenous receptor.

In some cases, the subject is a non-human subject. In some cases, the subject can be a variety of subjects capable of perceiving pain, with assays known in the art able to measure the perception of pain by the subject. Generally, such subjects are non-human. Generally, such subjects are “mammals” or “mammalian”, where these terms are used broadly to describe organisms which are within the class mammalia, including the orders carnivore (e.g., dogs and cats), rodentia (e.g., mice, guinea pigs and rats), and primates (e.g., chimpanzees and monkeys). In some cases, non-mammalian subjects can be used (e.g., Drosophila, C. elegans and zebrafish).

A screening method can be an in vitro cell-based screening method for identifying test compounds that modulate pain-associated signaling in a cell. In such cases, the method may comprise a) delivering a polynucleotide that encodes an activatable exogenous receptor to a target cell, to express the receptor in the target cell; b) contacting the cell with an effective amount of an agent that specifically activates the exogenous receptor, wherein activation of the exogenous receptor results in a quantifiable event; c) contacting the cell with the test compound; and d) determining if the test compound reduces or enhances the quantifiable event in the cell. In some cases, a quantifiable event is a signaling output, for example, the level of cAMP production, or the level of presynaptic glutamate released. Suitable cells for use in a subject in vitro screening method are known in the art and may include cells (e.g., neurons) derived from a variety of organisms (e.g., mice, rats, chimpanzees, and the like).

In some instances, a class of test compound that is of interest is a test compound that reduces the perception of pain. Perception of pain can be measured by any of the assays previously described. Such a test compound may reduce the perception of pain by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or more, compared to the level of pain perceived by the subject in the absence of the test compound. Such a test compound may reduce the level of the quantifiable event in the cell of an in vitro cell-based screening method by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or more, compared to the level of the quantifiable event in the absence of the test compound.

In other instances, a class of test compound that is of interest is a test compound that enhances the perception of pain. Perception of pain can be measured by any of the assays previously described. Such a test compound may enhance the perception of pain by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or more, compared to the level of pain perceived by the subject in the absence of the test compound. Such a test compound may increase the level of the quantifiable event in the cell of an in vitro cell-based screening method by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or more, compared to the level of the quantifiable event in the absence of the test compound.

In some cases, the present disclosure provides test compounds that can be further developed into therapeutic agents. For example, a test compound that reduces the perception of pain in a subject (e.g., by at least about 40%) can be further developed into a therapeutic agent for the treatment of pain.

The terms “candidate agent,” “test agent,” “agent,” “substance,” and “test compound” are used interchangeably herein. Test compounds encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Screening may be directed to known pharmacologically active compounds and chemical analogs thereof, or to new agents with unknown properties such as those created through rational drug design.

Test compounds may be small organic or inorganic compounds having a molecular weight of more than 50 and less than about 10,000 daltons, e.g., from about 50 daltons to about 100 daltons, from about 100 daltons to about 500 daltons, from about 500 daltons to about 1000 daltons, from about 1000 daltons to about 5000 daltons, or from about 5000 daltons to about 10,000 daltons. Test compounds may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Subject screening methods may include controls, where suitable controls include, e.g., a sample (e.g., a sample comprising the test subject) in the absence of the test compound. Generally, a plurality of assay mixtures is run in parallel with different test compounds concentrations to obtain a differential response to the various concentrations. Typically, one of these concentrations serves as a negative control, i.e. at zero concentration or below the level of detection.

Test compounds that have an effect in a subject screening method may be further tested for cytotoxicity, bioavailability, and the like, using well known assays. Test compounds that have an effect in a subject assay method may be subjected to directed or random and/or directed chemical modifications, such as acylation, alkylation, esterification, amidification, etc. to produce structural analogs. Such structural analogs include those that increase bioavailability, and/or reduced cytotoxicity. Those skilled in the art can readily envision and generate a wide variety of structural analogs, and test them for desired properties such as increased bioavailability and/or reduced cytotoxicity and/or ability to cross the blood-brain barrier.

A variety of other reagents may be included in the screening assay. These include reagents like salts, neutral proteins, e.g. albumin, detergents, etc., that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hour will be sufficient.

A test compound is assessed for any cytotoxic activity it may exhibit toward the subject used in a screening method of the present disclosure, using well-known assays, such as trypan blue dye exclusion, an MTT ([3-(4,5-dimethylthiazol-2-yl)-2,5-diphenyl-2 H-tetrazolium bromide]) assay, and the like. Test compounds that do not exhibit significant cytotoxic activity may be considered as candidate agents.

Any candidate agent identified can be further evaluated, for example, in a secondary screen to determine efficacy in other cell types, to determine cell type specific effects, and the like.

### Compositions and Kits

### Pharmaceutical Compositions

The present disclosure provides pharmaceutical compositions comprising a subject polynucleotide encoding an activatable exogenous receptor. Also provided are pharmaceutical compositions comprising an agent that specifically activates the exogenous receptor. The subject polynucleotides and agents can be incorporated into a variety of formulations for administration to a subject. More particularly, subject polynucleotides and agents can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. The formulations may be designed for administration via a number of different routes, including oral, buccal, rectal, parenteral, intraperitoneal, intradermal, transdermal, intracheal, etc., administration. In some cases the formulations can be administered through an implanted drug-infusion pump.

In pharmaceutical dosage forms, the subject polynucleotides and agents may be administered in the form of their pharmaceutically acceptable salts, or they may also be used alone or in appropriate association, as well as in combination, with other pharmaceutically active compounds. The following methods and excipients are merely exemplary and are in no way limiting.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and U.S. Pat. No. 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The subject polynucleotides and agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The subject polynucleotides and agents can be utilized in aerosol formulation to be administered via inhalation. The subject polynucleotides and agents of the present invention can be formulated into pressurized acceptable propellants such as dichlorodifluoromethane, propane, nitrogen and the like.

Furthermore, the subject polynucleotides and agents can be made into suppositories by mixing with a variety of bases such as emulsifying bases or water-soluble bases. The subject polynucleotides and agents of the present invention can be administered rectally via a suppository. The suppository can include vehicles such as cocoa butter, carbowaxes and polyethylene glycols, which melt at body temperature, yet are solidified at room temperature.

The subject polynucleotides and agents of this invention and their pharmaceutically acceptable salts which are active on topical administration can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference in its entirety. Such patches may be constructed for continuous, pulsatile, or on demand delivery of pharmaceutical agents.

Optionally, the pharmaceutical composition may contain other pharmaceutically acceptable components, such a buffers, surfactants, antioxidants, viscosity modifying agents, preservatives and the like. Each of these components is well-known in the art. See, for example, U.S. Pat. No. 5,985,310, the disclosure of which is herein incorporated by reference.

Other components suitable for use in the formulations of the present disclosure can be found in Remington's Pharmaceutical Sciences, Mace Publishing Company, Philadelphia, Pa., 17th ed. (1985). In an embodiment, the aqueous cyclodextrin solution further comprise dextrose, e.g., about 5% dextrose.

Dosage levels of pharmaceutical compositions comprising a subject polynucleotide of the order of from about 10⁹ vg/kg to 10¹² vg/kg are useful in representative embodiments. For example, a dosage level of the order of from about 10⁸ vg/kg to 10¹³ vg/kg is useful in representative embodiments, e.g., 10⁷ vg/kg, 10⁸ vg/kg, 10⁹ vg/kg, 10¹⁰ vg/kg, 10¹¹ vg/kg, 10¹² vg/kg, 10¹³ vg/kg, 10¹⁴ vg/kg, at least about 10⁷ vg/kg, at least about 10⁸ vg/kg, at least about 10⁹ vg/kg, at least about 10¹⁰ vg/kg, at least about 10¹¹ vg/kg, at least about 10¹² vg/kg, at least about 10¹³ vg/kg, at least about 10¹⁴ vg/kg. For example, a dosage level useful in representative embodiments can be from about 10⁸ vg/kg to 10⁹ vg/kg, can be from about 10⁹ vg/kg to 10¹⁰ vg/kg, can be from about 10¹⁰ vg/kg to 10¹¹ vg/kg, can be from about 10¹¹ vg/kg to 10¹² vg/kg.

Dosage levels of pharmaceutical compositions comprising a subject agent of the order of from about 0.01 mg/kg to 10 mg/kg are useful in representative embodiments. For example, a dosage level of the order of from about 0.01 mg/kg to 0.1 mg/kg, from about 0.1 mg/kg to 1 mg/kg, from about 1 mg/kg to 10 mg/kg, from about 0.05 to 0.5 mg/kg, from about 0.5 mg/kg to 5 mg/kg, from about 5 mg/kg to about 15 mg/kg, at least about 10 mg/kg is useful in representative embodiments.

For example, pain may be effectively treated by the administration of from about 10⁹ vg/kg to 10¹² vg/kg of a pharmaceutical composition comprising a subject polynucleotide, and administration of from about 0.01 mg/kg to 10 mg/kg of a pharmaceutical composition comprising a subject agent. Those of skill will readily appreciate that dose levels can vary as a function of the specific compound, the severity of the symptoms and the susceptibility of the subject to side effects. Dosages for a given pharmaceutical composition are readily determinable by those of skill in the art by a variety of means.

It is understood by those of skill in the art that the specific dose level for any particular subject will depend upon a variety of factors including the age, body weight, general health, sex, diet, time of administration, route of administration, rate of excretion, drug combination and the severity of the particular disease undergoing therapy.

As such, unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular composition employed and the effect to be achieved, and the pharmacodynamics associated with each compound in the subject host.

### Kits

Kits for modulating pain in a subject are also described in the present disclosure. In general, subject kits may comprise a first pharmaceutical composition comprising a polynucleotide encoding an activatable exogenous receptor; and a second pharmaceutical composition comprising an agent that specifically activates the exogenous receptor. In some cases, the first pharmaceutical composition comprises a polynucleotide encoding a DREADD, and the second pharmaceutical composition comprises a synthetic small molecule that specifically activates the DREADD. In some cases, the first pharmaceutical composition comprises a polynucleotide encoding a modified human muscarinic receptor 4, and the second pharmaceutical composition comprises clozapine-N-oxide.

A subject kit can include any combination of components and compositions for performing the methods of the present disclosure. The components of a subject kit can be present as a mixture or can be separate entities. In some cases, components are present as a lyophilized mixture. In some cases, the components are present as a liquid mixture. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

### Intraneural Injection:

6-8 week old mice were anesthetized with 2% isoflurane, the thigh region was cleaned and sterilized, and then the sciatic nerve was exposed. A 35 G needle was inserted into the nerve, and 2-5 μl of AAV6-hSyn-HA-hM4D-IRES-mCitrine was injected into the nerve, at the rate of 1 μl/min. The cavity was then closed, analgesics were administered, and the recovery of the mice was observed. In some experiments, mice were co-injected with 3 μl of AAV6-hSyn-ChR2-YFP, to allow for analysis of chemogenetic inhibition of optogenetically evoked pain response.

### Mechanical Withdrawal Testing:

Mice were tested 3-5 weeks after intraneural injection. Mice were placed on a von Frey apparatus, and allowed to habituate for 1 hour. Mechanical withdrawal thresholds were obtained using the up-down method of testing. Mice were then briefly anesthetized with isoflurane, and injected intra-peritoneally with 100 μl of clozapine-N-oxide (for an effective dose of 10 mg/kg) or 100 μl of saline. Post-injection testing was done in a blinded manner, with mechanical withdrawal thresholds collected at 45-120 minutes post-injection. Data were then unblinded, and statistical significance assessed using the Student's t-test.

### Thermal Withdrawal Testing:

Mice were tested 3-5 weeks after intraneural injection. Mice were placed on a Hargreaves apparatus, and allowed to habituate for 1 hour. Thermal withdrawal latencies were then obtained. Mice were then briefly anesthetized with isoflurane, and injected intra-peritoneally with 100 μl of clozapine-N-oxide (for an effective dose of 10 mg/kg) or 100 μl of saline. Post-injection testing was done in a blinded manner, with thermal withdrawal latencies collected at 45-120 minutes post-injection. Data were then unblinded, and statistical significance assessed using the Student's t-test.

### Optical Withdrawal Testing:

In mice that were co-injected with AAV6-hSyn-HA-hM4D-IRES-mCitrine and AAV6-hSyn-ChR2-YFP optical withdrawal testing was performed 2-3 weeks post injection. Mice were placed on a glass surface and allowed to habituate for 30-45 minutes. A 473 nm laser was connected to a fiber optic cable, and used to illuminate the paw regions of the mice at varying light intensities. Mice responded to the blue light with characteristic pain-signs, engaging in paw withdrawal, shaking, and licking. Light intensities were progressively reduced to determine the minimal threshold at which animals consistently engaged in a withdrawal response. This value was termed the ‘optical withdrawal threshold’. Due to variation in the effective illumination spot, the optical withdrawal threshold was measured in total illuminated output (in mW), rather than in mW/mm². Mice were then briefly anesthetized with isoflurane, and injected intra-peritoneally with 100 μl of clozapine-N-oxide (for an effective dose of 10 mg/kg) or 100 μl of saline. Post-injection testing was done in a blinded manner, with optical withdrawal thresholds collected at 60-90 minutes. Data were then unblinded, and statistical significance assessed using the Student's t-test.

### Example 1

A previously described viral expression strategy (Iyer et al., Nat. Biotechnol., 2014. 32:274-278) was adapted to specifically transduce primary afferent nociceptors with hM4D(Gi). Robust DREADD expression was observed, as assessed through expression of the fluorescent reporter mCitrine in small diameter primary afferent nociceptors. Expression characteristics were broadly similar to those previously described following intraneural injection of AAV6.

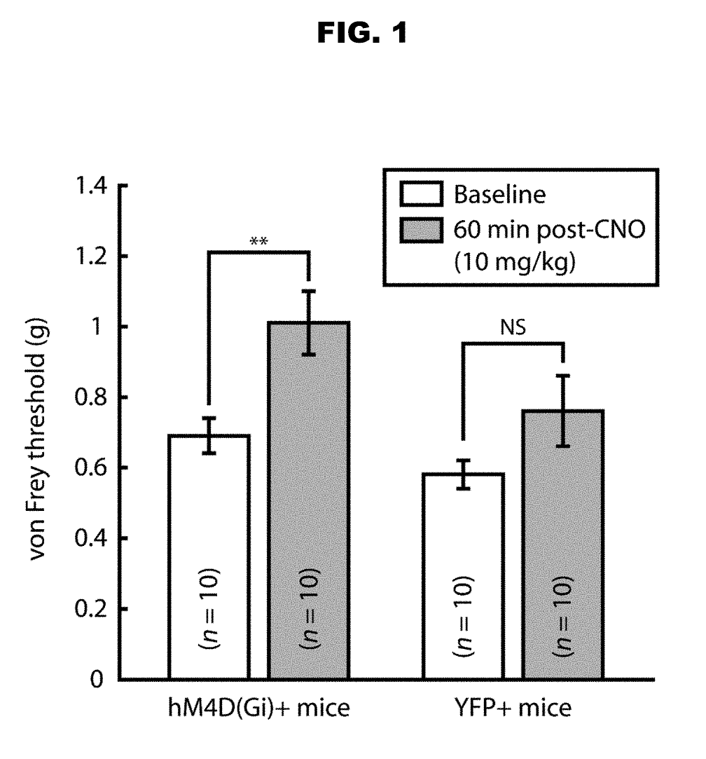

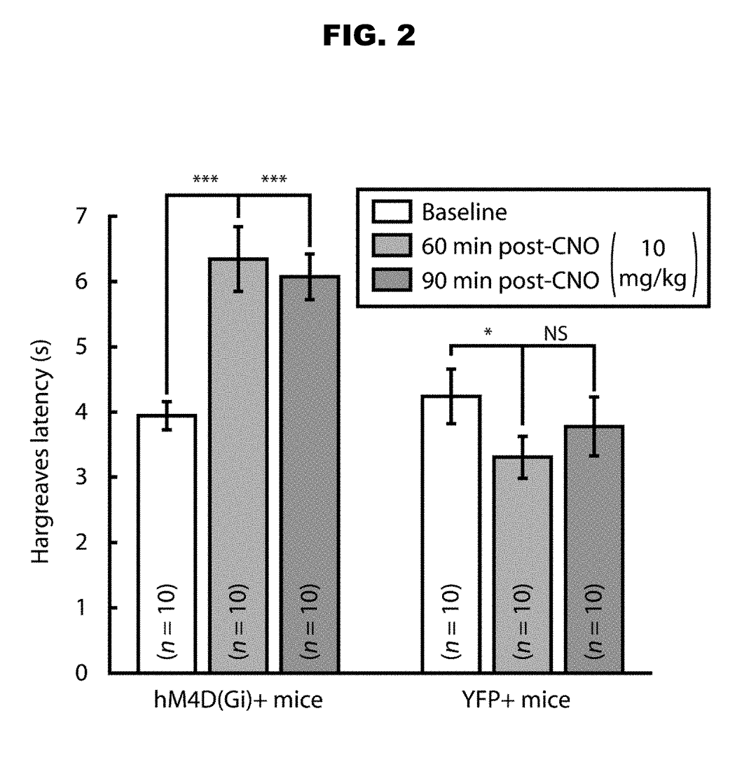

The effect of intraperitoneal clozapine-N-oxide administration was examined on mouse mechanical and thermal pain thresholds. In blinded experiments, it was observed that CNO robustly increased mechanical withdrawal thresholds in mice expressing the hM4D receptor (45% increase, effect size=1.3, P=0.02, n=10 paws) (FIG. 1). The effect size of inhibition that was observed was comparable to what has been previously reported with optogenetic inhibition of primary afferents using NpHR (0.802) (Iyer et al., Nat. Biotechnol., 2014. 32:274-278). Consistent with these previous results, chemogenetic inhibition of thermal sensation resulted in similarly strong inhibition of Hargreaves thresholds. Following CNO administration, a 61% increase in thermal thresholds at 60 minutes post-injection (effect size=1.903, n=10, P=0.000915) was observed, which remained stable at a 54% increase as late as 90 minutes post-injection (effect size=2.2245, n=10, P=0.0004) (FIG. 2). The effect size of the inhibition observed was again very similar to that previously observed with optogenetic inhibition of thermal perception using NpHR (2.05) (Iyer et al., Nat. Biotechnol., 2014. 32:274-278).

FIG. 1 demonstrates the chemogenetic suppression of mechanical withdrawal thresholds (von Frey thresholds) of hM4D(Gi) mice 60 min after administration of 10 mg/kg CNO.

FIG. 2 demonstrates the chemogenetic suppression of thermal withdrawal latencies in the Hargreaves test of hM4D(Gi) mice 60 or 90 min after administration of 10 mg/kg CNO.

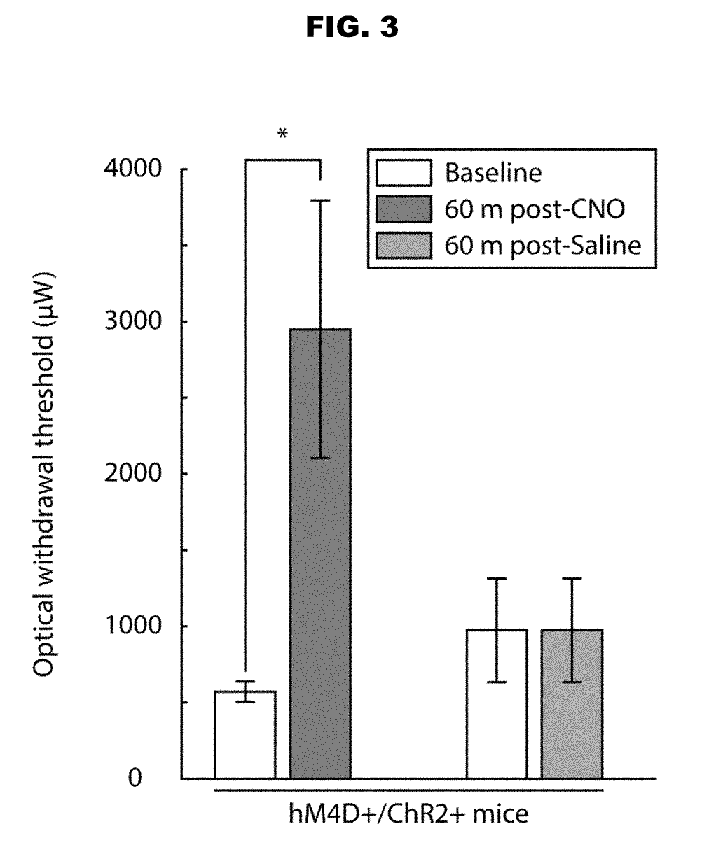

Whether optogenetic and chemogenetic approaches could be combined in the same system to allow for bidirectional control over pain perception was tested. Mice were injected with a mixture of AAV6-hSyn-ChR2-eYFP and AAV6-hSyn-HA-hM4D-IRES-mCitrine. Two weeks after injection, mouse pain sensitivity was assayed and it was observed that light intensities required to achieve a pain-related response were similar to those previously observed in mice injected only with AAV6-hSyn-ChR2-YFP. In blinded experiments, it was observed that following CNO administration, the required light intensity to achieve a pain-related response increased by 418% (effect size=1.603, n=5 mice, P=0.045) (FIG. 3), indicating that while the optogenetic stimulatory effect was ultimately stronger than the chemogenetic inhibitory effect, chemogenetic inhibition could significantly modulate optogenetic sensitivity. It was tested if other analgesic approaches could have a similar effect on the light intensity required to achieve a pain-related response. It was observed that a variety of different analgesics, including buprenorphine and gabapentin had strong effects on optical light sensitivity in blinded experiments.

FIG. 3 demonstrates the chemogenetic suppression of optical withdrawal thresholds in hM4D+/ChR2+ mice 60 min after administration of CNO.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the embodiments shown and described herein. Rather, the scope and spirit of present invention is embodied by the appended embodiments.

Notwithstanding the appended claims, the disclosure set forth herein is also described by the following clauses.

1. A method of modulating pain in a subject, the method comprising: a) delivering a polynucleotide that encodes an activatable exogenous receptor to a target neuron that mediates pain perception in the subject, to express the activatable exogenous receptor in the target neuron; b) administering to the subject an effective amount of an agent that specifically activates the exogenous receptor to alter the activity of the target neuron, thereby modulating pain in the subject.
2. The method of clause 1, wherein the activatable exogenous receptor is a mutant form of an endogenous receptor expressed by the target neuron.
3. The method of any one of clauses 1-2, wherein the polynucleotide is delivered via intraneural viral delivery.
4. The method of any one of clauses 1-3, wherein the polynucleotide is delivered via intrathecal, topical, intradermal, intra-dorsal root ganglion, or intravenous viral delivery.
5. The method of any one of clauses 1-4, wherein activation of the activatable exogenous receptor in (a) hyperpolarizes the target neuron.
6. The method of any one of clauses 1-5, wherein the activatable exogenous receptor is a modified human muscarinic receptor 4.
7. The method of any one of clauses 1-6, further comprising: delivering a second polynucleotide that encodes a light-activatable polypeptide to the target neuron, to express the light-activatable polypeptide in the target neuron, wherein activation of the light-activatable polypeptide depolarizes the target neuron; wherein activation of the activatable exogenous receptor in (a) hyperpolarizes the target neuron.
8. The method of any one of clauses 1-7, wherein the polynucleotide comprises the amino acid sequence of human muscarinic receptor 4 as set forth in SEQ ID NO:3 or 4.
9. The method of clause 8, wherein activation of the activatable exogenous receptor decreases cAMP levels in the target neuron by at least 10% to inhibit target neuron activity.
10. The method of any one of clauses 1-9, wherein the polynucleotide is delivered using an adeno-associated virus (AAV) vector.
11. The method of clause 10, wherein the polynucleotide further comprises adeno-associated virus type 6 (AAV6) sequence, AAV1, AAV2, AAV5 or AAV8 sequence.
12. The method of clause 10, wherein the polynucleotide sequence encoding the activatable exogenous reporter is in operable linkage with a human synapsin 1 promoter, a human Thy1 promoter, or a human calmodulin-dependent kinase II alpha (CaMKIIα) promoter.
13. The method of any one of clauses 1-12, wherein the agent is clozapine-N-oxide (CNO).
14. The method of clause 13, wherein CNO is administered within the range of 0.01 mg/kg to 10 mg/kg.
15. The method of any one of clauses 1-14, wherein the agent has an affinity for the activatable exogenous receptor of at least 0.01 μM.
16. The method of any one of clauses 1-15, wherein the agent is administered intraperitoneally.
17. The method of any one of clauses 1-15, wherein the agent is administered orally.
18. The method of any one of clauses 1-15, wherein the agent is administered via an implanted drug-infusion pump.
19. The method of any one of clauses 1-18, wherein modulating pain results in suppression of pain.
20. The method of any one of clauses 1-19, wherein the target neuron is an unmyelinated primary afferent neuron.
21. The method of clause 20, wherein the unmyelinated primary afferent neuron is an unmyelinated primary afferent nociceptor.
22. The method of any one of clauses 1-21, wherein the polynucleotide is delivered at a dose ranging from 10⁹ to 10¹² vg/kg.
23. The method of any one of clauses 1-22, wherein activating the exogenous receptor results in hyperpolarization of the target neuron.
24. The method of any one of clauses 1-22, wherein activating the exogenous receptor results in reduced presynaptic glutamate release.
25. A method of modulating activity of a neuron the method comprising: a) contacting the neuron with a polynucleotide that encodes an activatable exogenous receptor, under conditions sufficient to express the activatable exogenous receptor in the neuron; and b) contacting the neuron with an agent that specifically activates the activatable exogenous receptor thereby modulating the activity of the neuron.
26. The method of clause 25, wherein the polynucleotide is delivered using an adeno-associated virus (AAV) vector.
27. The method of any one of clauses 25-26, wherein the polynucleotide sequence encoding the activatable exogenous receptor is in operable linkage with a human synapsin 1 promoter, a human Thy1 promoter, or a human calmodulin-dependent kinase II alpha (CaMKIIα) promoter.
28. The method of any one of clauses 25-27, wherein the activatable exogenous receptor is a modified muscarinic receptor 4 and specifically binds the agent.
29. The method of any one of clauses 25-28, wherein the polynucleotide comprises the amino acid sequence of human muscarinic receptor 4 as set forth in SEQ ID NO:3 or 4.
30. The method of any one of clauses 25-29, wherein the polynucleotide further comprises adeno-associated virus type 6 (AAV6) sequence, AAV1, AAV2, AAV5 or AAV8 sequence.
31. The method of any one of clauses 25-30, wherein the polynucleotide is delivered at 10⁹-10¹² vg/kg.
32. The method of any one of clauses 25-31, wherein activation of the activatable exogenous receptor decreases cAMP levels in the target neuron by at least 10%.
33. The method of any one of clauses 25-32, wherein the agent is clozapine-N-oxide (CNO).
34. The method of any one of clauses 25-33, wherein the target neuron is an unmyelinated primary afferent neuron.
35. The method of clause 34, wherein the unmyelinated primary afferent neuron is an unmyelinated primary afferent nociceptor.
36. A screening method to assess whether a test agent modulates pain perception in a subject, the method comprising: a) delivering a polynucleotide that encodes an activatable exogenous receptor to a target neuron that mediates pain perception in the subject, to express the receptor in the target neuron; b) administering to the subject an effective amount of an agent that specifically activates the exogenous receptor; c) administering the test compound; and d) determining if the test compound modulates pain perception in the subject.
37. The screening method of clause 36, wherein the agent that specifically activates the exogenous receptor enhances pain perception in the subject animal, and the test compound reduces pain perception in the subject animal.
38. The screening method of any one of clauses 35-36, wherein the activatable exogenous receptor is a modified human muscarinic receptor 3 that specifically binds the agent (e.g., comprising SEQ ID NO: 1 or 2).
39. The screening method of any one of clauses 36-38, wherein the polynucleotide is delivered using an adeno-associated virus (AAV) vector.
40. The screening method of any one of clauses 36-39, wherein the polynucleotide sequence encoding the activatable exogenous receptor is in operable linkage with a human synapsin 1 promoter, a human Thy1 promoter, or a human calmodulin-dependent kinase II alpha (CaMKIIα) promoter.
41. The screening method of any one of clauses 36-40, wherein the agent that specifically activates the exogenous receptor is clozapine-N-oxide.
42. The screening method of any one of clauses 36-41, wherein pain perception in the subject is assayed by a mechanical withdrawal test.
43. The screening method of any one of clauses 36-41, wherein pain perception in the subject is assayed by a Hargreaves test.
44. A kit comprising: a first pharmaceutical composition comprising a polynucleotide encoding a mutant form of a human muscarinic receptor 4; and a second pharmaceutical composition comprising an agent that specifically activates the receptor.
45. The kit of clause 44, wherein the agent is clozapine-N-oxide.

What is claimed is:

1. A method of modulating pain in a subject, the method comprising:
a) delivering a polynucleotide that encodes an activatable exogenous receptor to a target neuron that mediates pain perception in the subject, to express the activatable exogenous receptor in the target neuron;
b) administering to the subject an effective amount of an agent that specifically activates the exogenous receptor to alter the activity of the target neuron, thereby modulating pain in the subject.

2. The method of claim 1, wherein the activatable exogenous receptor is a mutant form of an endogenous receptor expressed by the target neuron.

3. The method of claim 1, wherein the polynucleotide is delivered via intraneural viral delivery, intrathecal, topical, intradermal, intra-dorsal root ganglion, or intravenous viral delivery.

4. The method of claim 1, wherein activation of the activatable exogenous receptor in (a) hyperpolarizes the target neuron.

5. The method of claim 1, wherein the activatable exogenous receptor is a modified human muscarinic receptor 4.

6. The method of claim 5, further comprising:
delivering a second polynucleotide that encodes a light-activatable polypeptide to the target neuron, to express the light-activatable polypeptide in the target neuron, wherein activation of the light-activatable polypeptide depolarizes the target neuron;
wherein activation of the activatable exogenous receptor in (a) hyperpolarizes the target neuron.

7. The method of claim 1, wherein the agent is clozapine-N-oxide (CNO).

8. The method of claim 1, wherein the agent is administered intraperitoneally, orally or via an implanted drug-infusion pump.

9. The method of claim 1, wherein the target neuron is an unmyelinated primary afferent neuron.

10. The method of claim 1, wherein activating the exogenous receptor results in hyperpolarization of the target neuron.

11. The method of claim 1, wherein activating the exogenous receptor results in reduced presynaptic glutamate release.

12. A method of modulating activity of a neuron the method comprising:
a) contacting the neuron with a polynucleotide that encodes an activatable exogenous receptor, under conditions sufficient to express the activatable exogenous receptor in the neuron;
b) contacting the neuron with an agent that specifically activates the activatable exogenous receptor thereby modulating the activity of the neuron.

13. The method of claim 12, wherein the polynucleotide sequence encoding the activatable exogenous receptor is in operable linkage with a human synapsin 1 promoter, a human Thy1 promoter, or a human calmodulin-dependent kinase II alpha (CaMKIIα) promoter.

14. The method of claim 12, wherein the activatable exogenous receptor is a modified muscarinic receptor 4 and specifically binds the agent.

15. The method of claim 12, wherein activation of the activatable exogenous receptor decreases cAMP levels in the target neuron by at least 10%.

16. The method of claim 12, wherein the agent is clozapine-N-oxide (CNO).

17. The method of claim 12, wherein the target neuron is an unmyelinated primary afferent neuron.

18. A screening method to assess whether a test agent modulates pain perception in a subject, the method comprising:
a) delivering a polynucleotide that encodes an activatable exogenous receptor to a target neuron that mediates pain perception in the subject, to express the receptor in the target neuron;
b) administering to the subject an effective amount of an agent that specifically activates the exogenous receptor;
c) administering the test compound; and
d) determining if the test compound modulates pain perception in the subject.

19. The screening method of claim 18, wherein the activatable exogenous receptor is a modified human muscarinic receptor 3 that specifically binds the agent; and the agent that specifically activates the exogenous receptor is clozapine-N-oxide.

20. The screening method of claim 18, wherein pain perception in the subject is assayed by a mechanical withdrawal test or a Hargreaves test.


SEQUENCE LISTING (4 sequences)

SEQ ID NO: 1 - protein, 590 aa, Artificial Sequence
  Synthetic polypeptide
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 2 - protein, 590 aa, Artificial Sequence
  Synthetic polypeptide
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDCVASNASVMNLLVISFDRYFSITRPLTYRAKRTTKRAGVMIGLAWVISFVLWAPAILFWQYFVGKRTVPPGECFIQFLSEPTITFGTAIAGFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 3 - protein, 479 aa, Artificial Sequence
  Synthetic polypeptide
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDYVVSNASVMNLLIISFDRYFCVTKPLTYPARRTTKMAGLMIAAAWVLSFVLWAPAILFWQFVVGKRTVPDNQCFIQFLSNPAVTFGTAIAAFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTAR

SEQ ID NO: 4 - protein, 479 aa, Artificial Sequence
  Synthetic polypeptide
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDCVVSNASVMNLLIISFDRYFCVTKPLTYPARRTTKMAGLMIAAAWVLSFVLWAPAILFWQFVVGKRTVPDNQCFIQFLSNPAVTFGTAIAGFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTAR